(19) United States
(12) Patent Application Publication
(10) Pub. No.: US 2021/0308228 A1
(43) Pub. Date: Oct. 7, 2021

(54) METHOD FOR REDUCING THE OCCURRENCE OF THROMBOSIS OR THROMBOEMBOLISM

(71) Applicants: THE BOARD OF REGENTS OF THE UNIVERSITY OF TEXAS SYSTEM, Austin, TX (US); GRIFOLS SHARED SERVICES NORTH AMERICA, INC., Los Angeles, CA (US)

(72) Inventors: Jessica C. CARDENAS, Houston, TX (US); Charles E. WADE, Houston, TX (US); Yao-Wei WANG, Houston, TX (US); Bryan A. COTTON, Houston, TX (US); Criston OSBORN, Research Triangle Park, NC (US)

(21) Appl. No.: 17/146,912

(22) Filed: Jan. 12, 2021

Related U.S. Application Data
(60) Provisional application No. 63/005,250, filed on Apr. 4, 2020.

(51) Int. Cl. A61K 38/36; A61K 31/727; A61K 31/737

(52) U.S. Cl. CPC A61K 38/36; A61K 31/737; A61K 31/727

(57) ABSTRACT

The present disclosure refers to a method and compositions for reducing the occurrence of thrombosis or thromboembolism in a patient identified as being at risk thereof, the method comprising administering to the patient a therapeutically effective amount of one or more anticoagulant selected from the group consisting of unfractionated heparin, a low molecular weight heparin, a heparinoid, fondaparinux, idraparinux, and combinations thereof, and antithrombin III (ATIII).

### PRIORITY CLAIM

The present application claims priority to U.S. Provisional Application Ser. No. 63/005,250, filed Apr. 4, 2020, the entire contents of which are hereby incorporated by reference.

### BACKGROUND

The present disclosure is related to the field of pharmaceutical products. Certain embodiments herein relate to methods and compositions for reducing the occurrence of a thrombosis or a thromboembolism in a patient identified as being at risk thereof, the method comprising administering to the patient a therapeutically effective amount of one or more anticoagulant, and antithrombin III (ATIII).

### Description of the Related Art

Venous thromboembolism (VTE), a disorder that includes deep vein thrombosis (DVT) and pulmonary embolism (PE), is a prevalent complication in patients with polytraumatic injuries. VTE remains one of the most common preventable causes of in-hospital death in this population, despite extensive prophylactic efforts to mitigate risk [1, 2]. Up to 30% of patients on prophylaxis will experience a VTE while in the hospital, depending largely on patient acuity and degree of surveillance. These events result in aggressive interventions, prolonged hospital stays, and increased medical care costs [2, 3]. Three months after discharge, the rate of VTE in surviving trauma patients remains over 10% [4] and 30% of thromboembolic events in the trauma population overall occur after discharge [5]. These are not clinically insignificant events. In fact, Drake et al. recently showed that 10.8% of preventable or potentially preventable deaths after hospital discharge are due to massive PE [6].

While the exact pathophysiology of VTE in trauma patients is not fully understood, it is most certainly multifactorial and thought to arise secondarily to a combination of risk factors including aberrant coagulation activation, endothelial dysfunction, sustained inflammation, protracted immobility, massive transfusion, and mechanical ventilation [2]. Elevation in thrombin generation following trauma, a strong predictor of VTE in trauma patients [7], has been reported in several studies [7-11]. Thus, after hemorrhage control is achieved, restoring hemostatic homeostasis by limiting thrombin generation becomes critically important for preventing thromboembolic complications in recovering trauma patients.

To address this, the American Academy of Chest Physicians instituted standard of care recommendations for aggressive VTE prophylaxis in trauma patients which includes protocolized anticoagulation [12]. One of the most commonly used prophylactic anticoagulants utilized is enoxaparin (Lovenox), a low molecular weight heparin that can be monitored in-hospital by measuring anti-FXa levels. Enoxaparin works by potentiating the activity of the circulating anticoagulant, antithrombin III (AT). Enoxaparin binds to AT and increases its activity for inhibiting FXa and thrombin, thus downregulating coagulation. Despite increasingly aggressive institutional thromboprophylaxis protocols, 50-70% of trauma patients do not achieve the recommended anti-FXa target range (0.1-0.4 IU/mL) which indicates an altered responsiveness to enoxaparin anticoagulation [13, 14]. Such irresponsiveness has been associated with the risk of VTE. For example, Malinoski et al. showed an increased incidence of DVT among trauma and surgical patients who did not achieve the recommended anti-FXa range [15]. Even escalating doses of heparinoids appear ineffective since the incidence of VTE remains unchanged when enoxaparin doses are adjusted in response to low anti-FXa levels [16]. Sabbagh et al. first introduced the use of fresh frozen plasma (FFP) for the reversal of heparin resistance in the mid 1980′s in patients undergoing cardiopulmonary bypass [17].

Therefore, there is still the need to provide a composition and a method of treatment which helps to achieve the recommended anti-FXa target range, which indicates a good responsiveness to enoxaparin anticoagulation, and may provide a novel treatment strategy for safely improving heparin-based thromboprophylaxis.

### SUMMARY

The present inventors have surprisingly found that ex vivo treatment with ATIII, but not fresh frozen plasma (FFP), resulted in improved enoxaparin-mediated inhibition of thrombin generation, which increases the effectiveness of enoxaparin thromboprophylaxis. Thus, the present disclosure provides methods and compositions for reducing the occurrence of a thrombosis or a thromboembolism in a patient identified as being at risk thereof. In some embodiments, the method comprises administering to the patient a therapeutically effective amount of an anticoagulant selected from the group consisting of unfractionated heparin, a low molecular weight heparin, heparinoid, fondaparinux, idraparinuc, and combinations thereof, and antithrombin III (ATIII).

In some embodiments, the thrombosis is venous thrombosis. In some embodiments, said thromboembolism is venous thromboembolism (VTE).

In some embodiments, the patient identified as being at risk is selected from the group consisting of a physical trauma patient, a perioperative patient, a peripartum patient, patients with restricted mobility, cancer patients, and combination thereof. In some embodiments, the patient is a physical trauma patient, and the patient has been subject to blunt force trauma, penetrating trauma, or a combination thereof.

In some embodiments, the ATIII is administered at a concentration that increases the patient's ATIII levels to greater than about 1.0 IU/mL. In some embodiments, the ATIII is administered at a concentration that increases the patient's ATIII level to greater than about 1.2 IU/mL. In some embodiments, the ATIII is administered at a concentration that increases the patient's ATIII level to greater than about 1.3 IU/mL. In some embodiments, the ATIII is administered at a concentration that increases the patient's ATIII level to greater than about 1.4 IU/mL. In some embodiments, the ATIII is administered at a concentration that increases the patient's ATIII level to greater than about 1.5 IU/mL. In some embodiments, the ATIII is administered at a concentration that increases the patient's ATIII level to a range of about 1.5 to about 2.5 IU/mL. In some embodiments, the ATIII is administered at a concentration that increases the patient's ATIII level to a range of about 1.5 to about 2.0 IU/mL. In some embodiments, the ATIII is administered at a concentration that increases the patient's ATIII levels to a range of about 1.0 IU/mL to about 1.5 IU/mL.

In some embodiments, the ATIII is plasma-derived or recombinant ATIII.

In some embodiments, the low molecular weight heparin is selected from the group consisting of Bemiparin, Certoparin, Dalteparin, Enoxaparin, Nadroparin, Parnaparin, Reviparin, Tinzaparin, combination thereof, and/or pharmaceutically acceptable salts thereof. In some embodiments, the heparinoid is selected from the group consisting of Danaparoid, Dermatan sulphate, Sulodexide, combination thereof and/or pharmaceutically acceptable salts thereof. In some embodiments, the anticoagulant is a low molecular weight heparin, more preferably said low molecular weight heparin is Enoxaparin or a pharmaceutically acceptable salt thereof.

In some embodiments, the composition of the present disclosure is administered subcutaneously. In some embodiments, the composition comprising enoxaparin, or a pharmaceutically acceptable salt thereof, and ATIII is administered subcutaneously. In some embodiments, the therapeutically effective amount of the low molecular weight heparin is a daily dose of about 20 mg to about 180 mg, preferably from 20 mg to 40 mg. In some embodiments, enoxaparin can be administered in an amount of about 20 mg to about 180 mg per day, or from about 20 mg to about 40 mg per day. In some embodiments, the therapeutically effective amount of the low molecular weight heparin is from about 0.1 to about 2.5 mg/kg. In some embodiments, the therapeutically effective amount of the low molecular weight heparin is from 0.5 to about 1.5 mg/kg.

### BRIEF DESCRIPTION OF THE DRAWINGS

FIG. 1 shows the flow of admitted and enrolled patients in the study of the present disclosure. PE=pulmonary embolism.

FIG. 2 shows antithrombin (AT) levels in trauma patient plasma at baseline and following treatment with fresh frozen plasma (FFP) or AT concentrate. AT is presented as percent activity. FFP treatment was 30% by volume. AT was supplemented to 120, 150, 180, or 200% final concentration. Data are presented as means with standard deviation. The dotted line represents the limits of detection. * denotes p<0.05 compared to baseline following one-way ANOVA analysis with Bonferroni correction.

FIGS. 3A-3B show the levels of Anti-FXa and thrombin following enoxaparin treatment. Anti-FXa levels (A) and changes in peak thrombin (B) were measured in trauma patients following treatment of their plasma with enoxaparin in the presence or absence of FFP or AT supplementation. Delta peak represents the percent change of thrombin in treated plasma compared to untreated plasma at baseline. Data are presented as means with standard deviation. * denotes p<0.05 compared to enoxaparin alone following one-way ANOVA analysis with Bonferroni correction.

FIGS. 4A-4B show the levels of Anti-FXa and thrombin following enoxaparin treatment of PE vs no PE patients. Anti-FXa levels (A) and changes in peak thrombin (B) were measured in plasma from patients who did (white bar) or did not develop a PE (black bar) following treatment with enoxaparin in the presence or absence of FFP or AT supplementation. Data are presented as means with standard deviation. * denotes p<0.05 comparing “PE” and “No PE” patients after two-way ANOVA analysis with Sidak correction.

### DETAILED DESCRIPTION

Definitions

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts of the compound (or deuterated analog, stereoisomer, or mixture of stereoisomers) with inorganic acids or organic acids. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids.

The term “treatment” or “treating” means any treatment of a disease or disorder in a subject, such as a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, condition, disease, or disorder, thereby substantially averting onset of the ailment, condition, disease, or disorder.

As used herein, the term “suppressing” refers to prophylactic treatment of a patient in need thereof. The prophylactic treatment can be accomplished by providing an appropriate dose of a therapeutic agent to a subject suffering from an underlying cause of an ailment, condition, disease, or disorder, but substantially averting onset of symptoms of the ailment, condition, disease, or disorder.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein.

The term “therapeutically effective amount” refers to that amount typically delivered as a pharmaceutical composition, that is sufficient to effect treatment, as defined herein, when administered to a subject in need of such treatment. On the other hand, the term “composition” can be a kit of parts as well, and does not necessarily have to be both actives co-formulated within a unit dosage form.

As used herein, the term “a critically ill patient” refers to a patient who is admitted to the intensive care unit (ICU) and stays in the ICU for at least 12 hours. In some embodiments, the critically ill patient stays in the ICU for at least 24 hours. In some embodiments, the critically ill patient stays in the ICU for at least two days, three days or four days. In some embodiments, the critically ill patient remains admitted to the hospital after being discharged from the ICU.

The “critically ill” or “critically ill patient” may have one or more conditions including, but are not limited to:

1. Acutely decompensated heart failure, New York Heart Association (NYHA) class III or IV;

2. Acute respiratory failure without the need for prolonged (<=2 days) respiratory support;

3. Acute infection without septic shock;

4. Acute rheumatic disorders (including acute lumbar pain, sciatica, vertebral compression, acute arthritis of the legs, or an episode of inflammatory bowel disease) or

5. Cancer.

Risk factors for venous thromboembolism (VTE) include, but are not limited to:

a) Age >75 years;

b) Previous history of VTE that required anticoagulant therapy;

c) Expected marked immobilization>=3 days (Level 1—bedrest without bathroom privileges);

d) Obesity (Body Mass Index (BMI)>30 for men or 28.6 for women);

e) Varicose veins or chronic venous insufficiency;

f) Lower extremity paresis;

g) Central venous catheterization;

h) Hormone therapy (antiandrogen, estrogen or selective estrogen receptor modulators (SERMs));

i) Chronic heart failure;

j) Chronic respiratory failure;

k) Active collagen vascular disease;

1) Acute infectious disease contributing to current hospitalization;

m) Erythropoeisis stimulating agents;

n) Inflammatory bowel disease;

o) Venous compression (tumor, hematoma or arterial anomaly);

p) Nephrotic syndrome; and

q) Inherited or acquired thrombophilia,

r) or a combination thereof.

In some embodiments, a critically ill patient has any one or more of conditions 1 to 4 above and has either at least two venous thromboembolism (VTE) risk factors from a) to q) as outlined above or a D-dimer result of more than two times the upper limit of normal.

As used herein, the term “thrombosis” refers to the formation of a blood clot inside a blood vessel, obstructing the flow of blood through the circulatory system. In some embodiments, the thrombosis is “venous thrombosis” which is a blood clot that forms within a vein. The term “thromboembolism” refers to the formation in a blood vessel of a clot (thrombus) that breaks loose and is carried by the blood stream to plug another vessel. The clot may plug a vessel for example in the lungs (pulmonary embolism), brain (stroke), gastrointestinal tract, kidneys, legs, among others.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

The present disclosure provides methods and compositions for reducing the occurrence of a thrombosis or a thromboembolism in a patient identified as being at risk thereof. In some embodiments, the method comprises administering to the patient a therapeutically effective amount of an anticoagulant selected from the group consisting of unfractionated heparin, a low molecular weight heparin, heparinoid, fondaparinux, idraparinuc, and combinations thereof, and antithrombin III (ATIII). In some embodiments, the thrombosis is venous thrombosis. In some embodiments, said thromboembolism is venous thromboembolism (VTE).

In some embodiments, the patient identified as being at risk is selected from the group consisting of a physical trauma patient, a perioperative patient, a peripartum patient, patients with restricted mobility, cancer patients, sepsis patients, systemic inflammatory response syndrome patients, and combinations thereof.

In some embodiments, the patient is a physical trauma patient, and the patient has been subject to blunt force trauma, penetrating trauma, or a combination thereof.

Surprisingly, the present inventors have found that supplementation of an anticoagulant with ATIII, but not plasma, increased ATIII levels in patients improving anticoagulant, such as enoxaparin-mediated inhibition of thrombin generation.

In some embodiments, the patient is at risk for thromboembolic complications.

In some embodiments, the patient has moderate or severe restricted mobility and other risk factors for VTE.

In some embodiments, a critically ill patient or the patient being admitted to or cared for in an intensive care unit is one that is at risk of developing a venous thromboembolic disease. In some embodiments, the patient suffers from one or more of (a) acutely decompensated heart failure, (b) acute respiratory failure, (c) acute infection without septic shock, (d) an acute rheumatic disorder or (e) cancer. In some embodiments, the patient suffers from decreased mobility.

In some embodiments, the acutely decompensated heart failure is New York Heart Association (NYHA) class III or IV. In some embodiments, the acute respiratory failure is without the need for prolonged (<=2 days) respiratory support. In some embodiments, the acute infection is without septic shock. In some embodiments, the patient suffers from one or more acute rheumatic disorders (including acute lumbar pain, sciatica, vertebral compression, acute arthritis of the legs, or an episode of inflammatory bowel disease).

In some embodiments, the patient is at risk of developing a venous thromboembolic disease. In another embodiment, the patient suffers from decreased mobility. In another embodiment, the thrombosis is venous thrombosis.

In some aspects, thrombosis is a feature of an underlying disease or condition. Non-limiting examples of such disease or condition include acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, thromboembolic stroke, systemic embolism, ischemic stroke, venous thromboembolism, atrial fibrillation, non-valvular atrial fibrillation, atrial flutter, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the disease or condition is selected from the group consisting of embolic stroke, thrombotic stroke, venous thrombosis, deep venous thrombosis, acute coronary syndrome, and myocardial infarction.

In some embodiments, the ATIII is administered at a concentration that increases the patient's ATIII level to greater than about 1.2 IU/mL. In some embodiments, the ATIII is administered at a concentration that increases the patient's ATIII level to greater than about 1.3 IU/mL. In some embodiments, the ATIII is administered at a concentration that increases the patient's ATIII level to greater than about 1.4 IU/mL. In some embodiments, the ATIII is administered at a concentration that increases the patient's ATIII level to greater than about 1.5 IU/mL. In some embodiments, the ATIII is administered at a concentration that increases the patient's ATIII level to a range of about 1.2 to about 2.5 IU/mL. In some embodiments, the ATIII is administered at a concentration that increases the patient's ATIII level to a range of about 1.5 to about 2.0 IU/mL.

In some embodiments, the ATIII is plasma-derived or recombinant ATIII.

Enoxaparin

In some embodiments, enoxaparin is the sodium salt of enoxaparin. Enoxaparin sodium is a biological substance obtained by alkaline depolymerization of heparin benzyl ester derived from porcine intestinal mucosa. Its structure is characterized by a 2-O-sulfo-4-enepyranosuronic acid group at the non-reducing end and a 2-N,6-O-disulfo-D-glucosamine at the reducing end of the chain. About 20% (ranging between 15% and 25%) of the enoxaparin structure contains a 1,6 anhydro derivative on the reducing end of the polysaccharide chain. The average molecular weight is about 4500 daltons. A non-limiting example of enoxaparin sodium is commercialized with the brand name Lovenox®. Lovenox 100 mg/mL concentration contains 10 mg enoxaparin sodium (approximate anti-Factor Xa activity of 1000 IU [with reference to the W.H.O. First International Low Molecular Weight Heparin Reference Standard]) per 0.1 mL of Water for Injection.

Antithrombin III

Antithrombin (AT) is an alpha2-glycoprotein of molecular weight 58,000 which is normally present in human plasma at a concentration of approximately 12.5 mg/dL and is the major plasma inhibitor of thrombin. Inactivation of thrombin by AT occurs by formation of a covalent bond resulting in an inactive 1:1 stoichiometric complex between the two, involving an interaction of the active serine of thrombin and an arginine reactive site on AT. AT is also capable of inactivating other components of the coagulation cascade including factors IXa, Xa, XIa, and XIIa, as well as plasmin. A non-limiting example of AT is commercialized with the brand name Thrombate III® (Grifols Therapeutics LLC, USA).

The compositions of this disclosure may be in the form of tablets, capsules, lozenges, or elixirs for oral administration, suppositories, sterile solutions or suspensions or injectable administration, and the like, or incorporated into shaped articles. The method of administration will vary from subject to subject and be dependent upon such factors as the type of mammal being treated, its sex, weight, diet, concurrent medication, overall clinical condition, the particular compounds and/or salts employed, the specific use for which these compounds and/or salts are employed, and other factors which those skilled in the medical arts will recognize.

The compositions of the present disclosure can be prepared for storage or administration by mixing active agents having a desired degree of purity with physiologically acceptable carriers, excipients, stabilizers etc., and may be provided in sustained release or timed release formulations. Acceptable carriers or diluents for therapeutic use are well known in the pharmaceutical field.

In some embodiments, dosage formulations to be used for therapeutic administration are sterile. Sterility is readily accomplished by filtration through sterile membranes such as 0.2 micron membranes, or by other conventional methods. Formulations typically will be stored in lyophilized form or as an aqueous solution. The pH of the preparations of this disclosure typically will be between 3 and 11, or from 5 to 9, or from 7 to 8. Route of administration may be by injection, such as intravenously (bolus and/or infusion), subcutaneously, intramuscularly, or colonically, rectally, nasally or intraperitoneally. Other dosage forms such as suppositories, implanted pellets or small cylinders, aerosols, oral dosage formulations (such as tablets, capsules and lozenges) and topical formulations such as ointments, drops and dermal patches may be used. The sterile membranes may be desirably incorporated into shaped articles such as implants which may employ inert materials such as biodegradable polymers or synthetic silicones, for example, Silastic, silicone rubber or other polymers commercially available. Preferably, the composition of the present disclosure is administered subcutaneously.

### EXAMPLES

The present disclosure is further defined by reference to the following examples. It will be apparent to those skilled in the art that many modifications, both to compositions and methods, may be practiced without departing from the scope of the present disclosure.

The study was a single institution, retrospective cohort analysis of prospectively collected data conducted at Memorial Hermann Hospital in Houston, Tex., a Level-1 trauma center, and the University of Texas Health Science Center at Houston. Prior approval from the Institutional Review Board was obtained (HSC-GEN-12-0059) for the study to include adult patients (≥16 years of age) admitted between October 2012 and October 2016 who met the criteria for highest-level trauma team activation and for whom consent was obtained within 72 hours of admission from either the patient or a legally authorized representative. A waiver of consent was obtained if a patient was discharged or died within 24 hours. Patients were excluded if they were <16 years of age, pregnant, prisoners, had greater than 20% total body surface area burned, were taking pre-hospital anticoagulants, died within 24 hours, or consent was not obtained. Prospective power analysis estimated that 50 patients per group (with versus without thromboembolic events) would be sufficient to detect a 20% difference in thrombin generation using an alpha of 0.05 and a power of 0.90%.

Patient demographics, vital signs, standard laboratory values, mechanism and severity of injury, and pre-hospital fluid and/or blood product administration were collected upon admission to the emergency department. Outcomes (PE, hospital-, ventilator-, intensive care unit (ICU)-free days, mortality), and 24-hour blood product and fluid administration were collected from patient records. A matched-group design was used to compare patients with and without PEs based on age, gender, injury mechanism and injury severity. After all PE patients were identified, non-PE patients that were admitted during this time frame with similar age, gender, and injury mechanisms and severity to the PE group were selected. Mann-Whitney tests were performed to compare these characteristics between the PE and non-PE groups. If differences approaching p=0.06 were identified, new non-PE patients were selected and the comparison was re-run until the groups were appropriately matched.

PE Categorization

Since the institution routinely monitors for PE symptoms, without screening for DVT, VTE was defined as the development of a PE in this study. If patients present with even mild symptoms related to PE, such as cough or shortness of breath, or asymptomatic pulmonary emboli are identified incidentally during the course of examination for other indications, a standard contrast-enhanced computerized tomography angiogram (CTA) of the chest was performed to establish a diagnosis. All diagnoses of PE were established clinically using radiological criteria, and CTA derived chest images were reviewed by staff radiologists for contrast filling defects in the pulmonary vasculature. A PE diagnosis was then categorized based on location of the filling defect as either central (involvement of the left and/or right main pulmonary artery or one or more lobar arteries), or segmental/sub-segmental (involvement of segmental and/or sub-segmental arteries).

Sample Collection

Patient samples were collected upon hospital admission, prior to clinical administration of enoxaparin. In conjunction with blood collection for standard hospital laboratory tests, an additional 20 mL of blood was obtained for research purposes. Blood was transferred into vacutainer tubes containing 3.2% citrate and after 30 minutes was centrifuged at 3,200 rpm for 20 minutes at room temperature to obtain platelet poor plasma. Plasma samples were then aliquoted and stored at −80° C. until analysis. Plasma was also collected from 15 healthy and consented volunteers under a separate protocol (HSC-MS-09-0314). Volunteers were excluded if they were on an anticoagulant or antiplatelet drug, were pregnant, or had a history of cardiovascular disease. Healthy subjects had a median age of 31 (30, 35), were 50% male, and were 63% Caucasian. Fresh frozen plasma (FFP) from five donors was purchased from Gulf Coast Regional Blood Center. A pool of the five donors was used for all ex vivo experimentation.

Plasma Analysis

Citrated plasma samples were thawed immediately prior to use. First, plasma from healthy, uninjured volunteers was used to optimize ex vivo dosing of enoxaparin. The ex vivo dose of enoxaparin needed to achieve 50% inhibition of thrombin generation was found to be 0.13 IU/mL, similar to that previously identified in the literature [22-24]. Given that the inventors spiked plasma with an enoxaparin dose of 0.13 IU/mL, our target anti-FXa level in response to enoxaparin was defined as 0.13 IU/mL. The responses to enoxaparin between healthy uninjured volunteers and trauma patients were compared by treating all plasma samples ex vivo with 0.13 IU/mL of enoxaparin. Trauma patient samples were additionally supplemented with AT using either FFP at a dose of 30% volume or AT concentrate in the form of THROMBATE III®, provided by Grifols, up to 80%, 120%, 150%, 180%, and 200% final concentration based on baseline values. This was done in an effort to improve patient plasma responses to enoxaparin to the levels observed in healthy controls. Similar to our previous work, the inventors chose FFP at a dose of 30% volume to model the effects of transfusing six units of plasma [25]. Calculations were based on the activity (IUs) provided by the manufacturer for a given lot of THROMB ATE III® (approximately 500 IUs/10 mL) and the patient's baseline AT activity levels. AT supplementation was confirmed by measuring AT activity using ACL-TOP. Thrombin generation and anti-FXa levels were measured to assess the response to enoxaparin and supplementation with AT. Healthy volunteer plasma served as an uninjured control for normal AT, anti-FXa, and thrombin generation levels before and after treatment with enoxaparin.

AT activity and anti-FXa levels were determined chromogenically using an ACL-TOP Coagulation Analyzer (Instrumentation Laboratory, Bedford Mass.). Thrombin generation was measured using a calibrated automated thrombogram (CAT) (Thermo Fisher Scientific, Waltham Mass.) as previously described [8]. Relevant parameters resulting from CAT analysis include lag time (the time to initiation of thrombin generation; min), peak (the highest amount of thrombin generated; nM), time to peak [ttpeak] (the time it takes to achieve the peak; min), endogenous thrombin potential [ETP] (the total amount of thrombin generated over time; nM), and rate (the velocity of thrombin generation dictated by the slope of the thrombin generation curve; nM/min).

Statistics

All analyses were performed using GraphPad Prism 6 (La Jolla, Calif.). For multiple comparisons, one-way or two-way ANOVA tests were done with Bonferroni or Sidak corrections, respectively. Mann-Whitney tests were performed where indicated. Data were considered significant at p<0.05.

Results

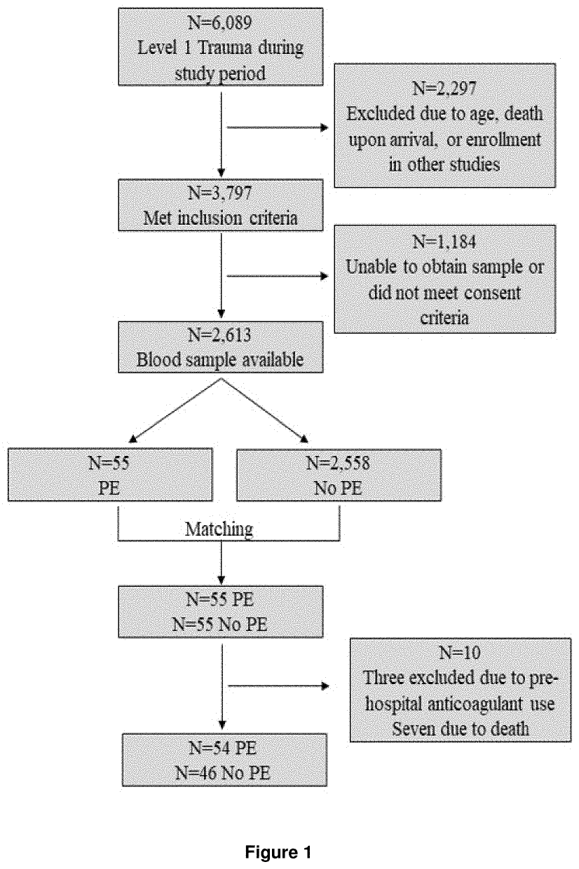

A consort diagram describing enrollment of the patient cohort is provided in FIG. 1. During the study period, 6,089 highest-level trauma activations were received at our institution.

Of those, 3,797 met the criteria for inclusion in our study. Plasma samples were available in our biorepository for 2,613 of these patients. A total of 55 patients with PE were identified during the study period. Of these patients, three were excluded for pre-hospital anticoagulant use and seven were excluded due to death from non-survivable traumatic brain injury. This left 100 patients for complete analysis (46 without PE and 54 with PE). The median time to PE diagnosis was hospital day 9 (4, 14). Among those patients who developed a PE, 68.5% were symptomatic and 31.5% were asymptomatic. Most PEs were located in segmental or sub-segmental arteries (67%), with the remainder located centrally (33%). Approximately 85% of PE patients received thrombosis-related interventions that included thrombectomy, tPA infusion, inferior vena cava (IVC) filter placement, unfractionated heparin drip, or another anticoagulant. Only 8 patients (15%) were maintained on enoxaparin alone. Patient demographics, injury types/severity, and outcomes are summarized in Table 1. There were no apparent differences in demographics or injury type and severity between patients who did versus those who did not develop a PE. However, those who developed a PE received significantly greater volumes of prehospital crystalloids and units of blood products during transport and over the first 24 hours of their hospital stay (all p<0.05). Furthermore, patients who developed a PE had significantly fewer ventilator-, ICU-, and hospital-free days (all p<0.01), although the overall in-hospital mortality rates were the same (8.5% vs 9.3%; p>0.05). Finally, while no differences in the rate of pelvic fractures were observed, patients who developed PE had a significant increase in the rate of lower limb fracture (28% vs 11%; p<0.05).

TABLE 1
Patient demographics, injury, resuscitation volumes and outcomes in
patients with and without pulmonary embolism (PE). Median and interquartile
range (IQR) values are reported. Mann-Whitney tests were performed
to determine statistical significance between groups.

| | | | |
|---|---|---|---|
| All | PE | No PE | |
| (N = 100) | (N = 54) | (N = 46) | P-value |

Demographics

| | | | | | | | |
|---|---|---|---|---|---|---|---|
| Age, years | 46 | (33, 54) | 47 | (35, 55) | 43 | (28, 53) | 0.19 |
| Male, n (%) | 79 | (79%) | 42 | (78%) | 37 | (80%) | 0.75 |

Injury

| | | | | | | | |
|---|---|---|---|---|---|---|---|
| Blunt, n (%) | 83 | (83%) | 47 | (87%) | 36 | (78%) | 0.24 |
| GCS | 13 | (3, 15) | 14 | (3, 15) | 8 | (3, 15) | 0.24 |
| w-RTS | 6.9 | (4.1, 7.8) | 7.1 | (4.1, 7.8) | 6.0 | (4.1, 7.8) | 0.57 |
| Head AIS | 2 | (0, 4) | 0 | (0, 3) | 2 | (0, 4) | 0.17 |
| ISS | 17 | (13, 26) | 19 | (13, 26) | 17 | (12, 25) | 0.38 |
| Pelvic Fracture, n (%) | 14 | (14%) | 8 | (15%) | 6 | (13%) | 0.80 |
| Lower Limb Fracture, n (%) | 20 | (20%) | 15 | (28%)* | 5 | (11%) | <0.05 |
| Base Excess | −3 | (−6, 0) | −3 | (−6, 0) | −3 | (−6, 0) | 0.98 |

Resuscitation Volumes

| | | | | | | | |
|---|---|---|---|---|---|---|---|
| Pre-hospital Crystalloid, mL | 0 | (0, 220) | 200 | (0, 500)* | 0 | (0, 0) | <0.01 |
| Pre-hospital RBC, units | 0 | (0, 0) | 0 | (0, 0)* | 0 | (0, 0) | 0.04 |
| Pre-hospital Plasma, units | 0 | (0, 0) | 0 | (0, 0)* | 0 | (0, 0) | <0.01 |
| 24 Hour RBC, units | 0 | (0, 2) | 1 | (0, 6)* | 0 | (0, 0) | <0.01 |
| 24 Hour Plasma, units | 0 | (0, 3) | 1 | (0, 6)* | 0 | (0, 1) | <0.01 |
| 24 Hour Platelets, units | 0 | (0, 0) | 0 | (0, 6)* | 0 | (0, 0) | <0.01 |

Outcomes

| | | | | | | | |
|---|---|---|---|---|---|---|---|
| Ventilator-free days | 27 | (19, 29) | 19 | (15, 27)* | 29 | (26, 30) | <0.01 |
| ICU-free days | 24 | (8, 28) | 17 | (1, 25)* | 27 | (21, 29) | <0.01 |
| Hospital-free days | 12 | (0, 20) | 0 | (0, 12)* | 20 | (15, 25) | <0.01 |
| 30-Day Mortality, n (%) | 8 | (8%) | 5 | (9.3%) | 3 | (6.5%) | 0.62 |

However, the presence of such fractures did not affect the pertinent outcomes of this study such as AT levels, rate of AT deficiency, thrombin generation, or responsiveness to enoxaparin, with or without AT supplementation.

Effects of Enoxaparin on Healthy Versus Trauma Patient Plasma.

Similar to previous reports (7), baseline thrombin generation was significantly elevated in trauma patients compared to healthy donors as evidenced by shortened lag times, increased peak, reduced time to peak, and increased rates of thrombin generation (Table 2).

TABLE 2
Antithrombin III (AT) levels, anti-FXa levels, and thrombin
generation values in plasma from healthy donors and trauma
patients at baseline and following treatment with enoxaparin
(0.13 U/mL). Number with percentage or median and IQR values
are reported. Mann-Whitney tests were performed to determine
statistical significance between groups.

| | | |
|---|---|---|
| Healthy Donors | Trauma Patients | P- |
| (N = 15) | (N = 100) | value |

Baseline

| Rate AT < 80% | 0 | | 20 | (20%) | |
|---|---|---|---|---|---|
| AT (%) | 114 | (109, 118) | 91 | (78, 103)* | <0.01 |
| Lag time (min) | 5.0 | (4.3, 5.7) | 4.3 | (3.7, 5.0)* | <0.05 |
| Peak (nM) | 258 | (153, 304) | 302 | (254, 345)* | <0.01 |
| ttPeak (min) | 8.3 | (7.0, 9.7) | 6.6 | (6.0, 7.4)* | <0.01 |
| ETP (nM) | 1459 | (1155, 1649) | 1492 | (1237, 1713) | 0.38 |
| Rate (nM/min) | 67.8 | (37.2, 107.7) | 129 | (106, 156)* | <0.01 |

Enoxaparin

| | | | | | |
|---|---|---|---|---|---|
| Anti FXa | 0.16 | (0.14, 0.17) | 0.13 | (0.12, 0.16)* | <0.01 |
| levels (IU/mL) | | | | | |
| Rate Anti | 15 | (100%) | 59 | (59%)* | <0.01 |
| FXa ≥ 0.13 | | | | | |
| Delta | 6.6 | (−4.3, 23) | 7.9 | (0, 11) | 0.90 |
| Lagtime (%) | | | | | |
| Delta Peak (%) | −55 | (−66, −36) | −24 | (−32, −16)* | <0.01 |
| Delta ttPeak (%) | 17 | (3.5, 36) | 12 | (7.3, 17) | 0.20 |
| Delta ETP (%) | −43 | (−48, −22) | −14 | (−20, −9)* | <0.01 |
| Delta Rate (%) | −65 | (−82, −57) | −39 | (−51, −27)* | <0.01 |

Trauma patients also displayed significantly lower anti-FXa levels after enoxaparin treatment compared to healthy controls. In response to enoxaparin treatment, all healthy donors achieved a target anti-FXa response of 0.13 IU/mL compared to 59% of trauma patients (p<0.01). Compared to healthy controls, trauma patients demonstrated significantly reduced inhibition of thrombin generation following treatment with enoxaparin as evidenced by reduced peak thrombin inhibition, reduced endogenous thrombin potential (ETP) inhibition, and reduced prolongation of the rate of thrombin production.

Supplementation of Enoxaparin with FFP or AT

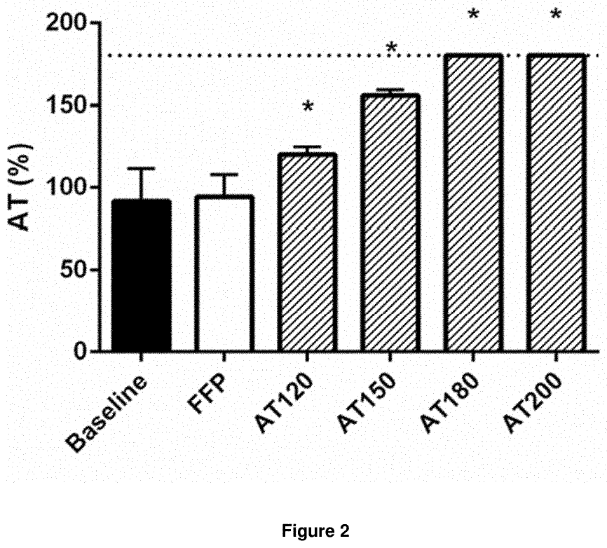

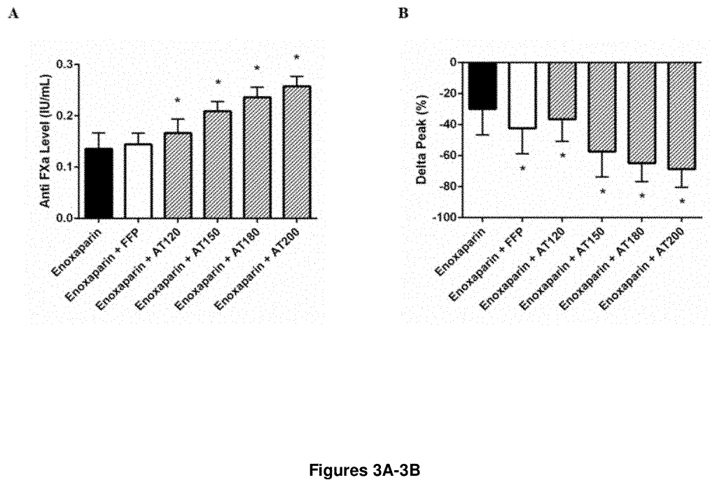

To determine if supplementation with either FFP or AT would increase AT levels and improve the response to enoxaparin, trauma patient plasma was treated ex vivo with enoxaparin (0.13 IU/mL) in addition to either FFP (30% volume) or AT (final concentration of 120-200%) (FIG. 2). FFP had a baseline AT level of 98 (96, 102). While treatment with FFP had no effect on AT levels, AT supplementation significantly increased baseline AT levels (limit of detection=180%). Treatment with FFP did not improve anti-FXa levels from enoxaparin alone; however, AT-supplemented enoxaparin increased anti-FXa levels in a dose dependent manner (FIG. 3A: all doses p<0.05).

FFP treatment improved the rate of therapeutic response from 59% to 80%; whereas, supplementation with AT to 120% resulted in 92% of trauma patient plasma reaching a therapeutic anti-FXa level. This increased to 100% after AT supplementation was raised to 150% or greater. In addition, while determining whether FFP or AT supplementation could improve the ability of enoxaparin to inhibit thrombin generation, the inventors found that both FFP and AT were able to significantly increase peak thrombin inhibition compared to enoxaparin alone (all p<0.05). The greatest inhibition was observed at AT levels of 150-200% (FIG. 3B).

Effects of Enoxaparin in Plasma from Patients with and without PE.

Baseline AT activity was similar between groups with no difference in the incidence of AT deficiency (Table 3). However, when comparing thrombin generation, patients who developed PE had significantly higher peaks (13%) and faster rates (24%) of thrombin generation at baseline compared to those without a PE. Moreover, patients who developed a PE had a significantly reduced response to enoxaparin ex vivo as measured by both anti-FXa levels and changes in thrombin generation. Anti-FXa levels in response to enoxaparin were significantly higher in plasma from patients who did not develop PE compared to those who did (0.14 vs 0.13 IU/mL; p<0.05). Ex vivo treatment with enoxaparin resulted in a median 30.6% reduction in peak thrombin in patients who did not develop PE; however, only a 20.7% reduction in peak thrombin was detected in those who did develop PE (p<0.01).

TABLE 3
AT levels and thrombin generation values in plasma from patients
with and without PE at baseline and following treatment with
enoxaparin (0.13 U/mL). Number with percentage or median and
IQR values are reported. Mann-Whitney tests were performed
to determine statistical significance between groups.

| | | |
|---|---|---|
| PE | No PE | P- |
| (N = 54) | (N = 46) | value |

Baseline

| | | | | | |
|---|---|---|---|---|---|
| Rate AT < 80% | 11 | (20.4%) | 9 | (19.6%) | 0.92 |
| AT (%) | 90 | (77, 99) | 93 | (78, 113) | 0.24 |
| Lag time (min) | 4.3 | (3.8, 4.8) | 4.2 | (3.7, 5.5) | 0.79 |
| Peak (nM) | 322 | (272, 351)* | 286 | (241, 335) | <0.05 |
| ttPeak (min) | 6.6 | (5.8, 7.2) | 6.6 | (6.0, 7.8) | 0.58 |
| ETP (nM) | 1524 | (1313, 1728) | 1489 | (1123, 1689) | 0.12 |
| Rate (nM/min) | 145 | (112, 161)* | 118 | (94, 150) | <0.05 |

Enoxaparin

| | | | | | |
|---|---|---|---|---|---|
| Anti FXa | 0.13 | (0.11, 0.14)* | 0.14 | (0.12, 0.17) | <0.05 |
| levels (IU/mL) | | | | | |
| Rate Anti | 24 | (44%) | 26 | (57%) | 0.23 |
| FXa ≥ 0.13 | | | | | |
| Delta | 7.9 | (0, 10.6) | 6.6 | (0, 13.1) | 0.74 |
| Lagtime (%) | | | | | |
| Delta Peak (%) | −20.7 | (−29.7, −15.3)* | −30.6 | (−37.8, −18.4) | <0.01 |
| Delta ttPeak (%) | 10.7 | (6.4, 15.0)* | 13.0 | (9.6, 20.4) | <0.05 |
| Delta ETP (%) | −13.2 | (−18.5, −8.8) | −15.6 | (−23.3, −11.3) | 0.10 |
| Delta Rate (%) | −34.2 | (−43.7, −26.1 * | −45.4 | (−55.3, −29.9) | <0.01 |

Supplementation of Enoxaparin with AT in PE Patient Plasma

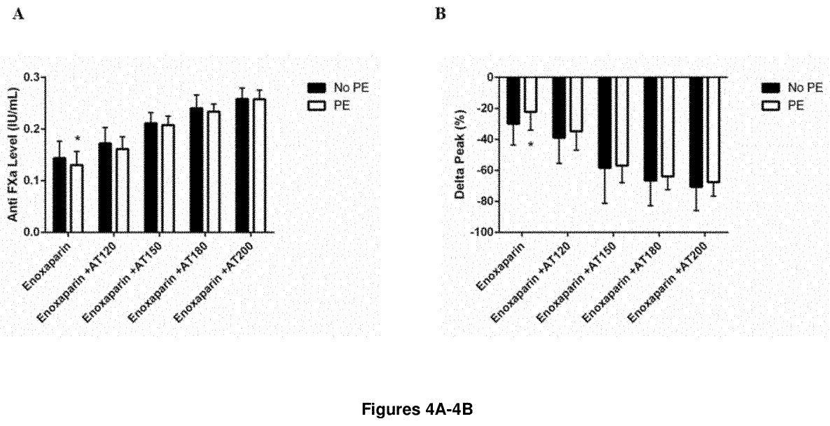

In order to determine the effects of supplementation with AT on the ex vivo response to enoxaparin, plasma from both PE and no PE patients was treated with enoxaparin and AT and changes in anti-FXa and thrombin inhibition were observed. AT significantly increased anti-FXa levels and reduced peak thrombin generation at all doses in plasma from all patients tested. PE patient plasma demonstrated lower anti-FXa levels (FIG. 4A) and reduced peak thrombin inhibition (FIG. 4B) compared to No PE patients with enoxaparin alone; however, these differences in enoxaparin responses were no longer apparent when AT supplementation was 120% or above.

Discussion

Persistent rates of VTE in the trauma patient population, in spite of aggressive, protocolized thromboprophylaxis, are a significant healthcare concern that necessitates new and evidence-based treatment strategies. AT concentrate is an FDA-approved, clinically available therapeutic used to treat congenital AT deficiency. To date, there is little data to show the potential role of AT supplementation in improving VTE prophylaxis in trauma patients. Here, the inventors demonstrate that plasma samples from severely injured patients, particularly from those who develop PE, exhibit reduced sensitivity to enoxaparin and that supplementation with AT improves enoxaparin-mediated thrombin inhibition ex vivo.

Enoxaparin and other low molecular weight heparin molecules are the most commonly utilized prophylactic agents in critical care patients during recovery. Enoxaparin increases both anti-FXa levels and AT inhibition of thrombin, making it a vital co-factor in preventing VTE. AT deficiency is commonly defined by hematologic standards as <80% activity with normal levels ranging between 80-120%. According to this definition, admission AT deficiency occurred in approximately 20% of trauma patients enrolled in this study which is similar to the rates reported by others [3]. While the majority of patients had AT levels within normal limits, only half of all patient plasma samples achieved a target anti-FXa level of 0.13 IU/mL following treatment with enoxaparin.

The inventors have previously shown that, compared to healthy subjects, trauma patients demonstrate profoundly elevated thrombin generation [8], a known risk factor for VTE [7]. Indeed, marked elevations were detected in this study as well. It is possible that in the setting of such excessive and protracted thrombin generation, circulating levels of AT are not adequate to facilitate prophylactic anticoagulation with enoxaparin. According to our findings, it appears that supraphysiologic levels of ATIII may be necessary to compete with such high levels of thrombin generation. Here, the inventors show that supplementation of trauma patient plasma with AT up to 120% raised enoxaparin effectiveness from 59% to 92% and that increasing AT above 120% optimized enoxaparin responses in terms of both anti-FXa levels and inhibition of thrombin generation. A 100% maximum effectiveness was attained when AT levels were raised to 150%. These findings are in agreement with other reports in the literature. Animal models of sepsis have shown that high doses of AT (˜160%) were most beneficial at mitigating hypercoagulability, reducing inflammation, and protecting from organ damage [26]. While maintaining ATIII activity at such high levels may not be feasible for all trauma patients, it may be appropriate to screen patients for potential responsiveness following the routine evaluation of their anti-FXa levels. Those who do not achieve the therapeutic range may be the best candidates for ATIII therapy. This could also provide an effective and safe alternative to hazardous interventions for patients with segmental or sub-segmental thrombi.

In spite of similar AT levels upon admission, patients who subsequently developed a PE had significantly reduced responses to enoxaparin compared to those who did not, a defect that was corrected through AT supplementation. This finding suggests that while there could be other mechanisms for enoxaparin resistance not evaluated here, modifying AT levels was nonetheless an effective intervention for increasing plasma enoxaparin sensitivity.

While FFP has been used in the past to treat heparin resistance [17, 28], the inventors did not find it to have significant effects here which is in agreement with previous work [18]. Even at 30% volume, equivalent to approximately six units or ˜20 ml/kg in a 70 kg adult, FFP had no effect on AT levels or the anti-FXa response to enoxaparin. FFP was, however, able to significantly reduce thrombin generation over enoxaparin alone. This is likely due to the endogenous AT in addition to other circulating anticoagulants present in balanced FFP that affect thrombin production such as tissue factor pathway inhibitor, protein C, protein S and thrombomodulin. Thus, administration of FFP may serve to mitigate thrombin generation by diluting endogenous hypercoagulable plasma with balanced plasma as previously shown [25]; however, our data suggest that it has very little direct effect on improving PE prophylaxis.

In summary, trauma is associated with reduced AT levels, increased thrombin generation, and reduced responsiveness to enoxaparin. Thrombin generation was higher and response to enoxaparin lower in patients who developed PE, and ex vivo treatment with AT, but not FFP, resulted in improved enoxaparin-mediated inhibition of thrombin generation.

Abbreviations

AIS, Abbreviated Injury Scale; ANOVA, Analysis of variance; AT, Antithrombin III; CAT, Calibrated automated thrombogram; CTA, Computerized tomography angiogram; DVT, Deep vein thrombosis; ETP, Endogenous thrombin potential; FFP, Fresh frozen plasma; FXa, Coagulation Factor Xa; GCS, Glasgow Coma Scale; ICU, Intensive care unit; ISS, Injury Severity Score; Kg, Kilogram; PE, Pulmonary embolism; RBC, Red Blood Cells; RPM, Revolutions per minute; ttPeak, Time to Peak; VTE, Venous thromboembolism; w-RTS, Weighted Revised Trauma Score.

### REFERENCES

[1] J. A. Acosta, J. C. Yang, R. J. Winchell, R. K. Simons, D. A. Fortlage, P. Hollingsworth-Fridlund, D. B. Hoyt, Lethal injuries and time to death in a level I trauma center, Journal of the American College of Surgeons 186(5) (218) 528-33.

[2] S. R. Hamada, C. Espina, T. Guedj, R. Buaron, A. Harrois, S. Figueiredo, J. Duranteau, High level of venous thromboembolism in critically ill trauma patients despite early and well-driven thromboprophylaxis protocol, Annals of intensive care 7(1) (2017) 97.

[3] C. R. Connelly, P. Y. Van, K. D. Hart, S. G. Louis, K. A. Fair, A. S. Erickson, E. A. Rick, E. C. Simeon, E. M. Bulger, S. Arbabi, J. B. Holcomb, L. J. Moore, M. A. Schreiber, Thrombelastography-Based Dosing of Enoxaparin for Thromboprophylaxis in Trauma and Surgical Patients: A Randomized Clinical Trial, JAMA surgery 151(10) (2016) e162069.

[4] L. N. Godat, L. Kobayashi, D. C. Chang, R. Coimbra, Can we ever stop worrying about venous thromboembolism after trauma?, The journal of trauma and acute care surgery 78(3) (2015) 475-80; discussion 480-1.

[5] M. P. DeWane, K. A. Davis, K. M. Schuster, A. A. Maung, R. D. Becher, Venous Thromboembolism-Related Readmission in Emergency General Surgery Patients: A Role for Prophylaxis on Discharge?, Journal of the American College of Surgeons (2018).

[6] S. A. Drake, J. B. Holcomb, Y. Yang, C. Thetford, L. Myers, M. Brock, D. A. Wolf, S. Cron, D. Persse, J. McCarthy, L. Kao, S. R. Todd, B. J. Naik-Mathuria, C. Cox, R. Kitagawa, G. Sandberg, C. E. Wade, Establishing a Regional Trauma Preventable/Potentially Preventable Death Rate, Annals of surgery (2018).

[7] M. S. Park, G. M. Spears, K. R. Bailey, A. Xue, M. J. Ferrara, A. Headlee, S. K. Dhillon, D. H. Jenkins, S. P. Zietlow, W. S. Harmsen, A. A. Ashrani, J. A. Heit, Thrombin generation profiles as predictors of symptomatic venous thromboembolism after trauma: A prospective cohort study, The journal of trauma and acute care surgery 83(3) (2017) 381-387.

[8] J. C. Cardenas, E. Rahbar, M. J. Pommerening, L. A. Baer, N. Matijevic, B. A. Cotton, J. B. Holcomb, C. E. Wade, Measuring thrombin generation as a tool for predicting hemostatic potential and transfusion requirements following trauma, The journal of trauma and acute care surgery 77(6) (2014) 839-45.

[9] N. M. Dunbar, W. L. Chandler, Thrombin generation in trauma patients, Transfusion 49(12) (2009) 2652-60.

[10] C. E. Wade, L. A. Baer, J. C. Cardenas, L. E. Folkerson, K. Nutall-Aurora, B. A. Cotton, N. Matijevic, J. B. Holcomb, J. M. Cross, T. Huzar, Upon admission coagulation and platelet function in patients with thermal and electrical injuries, Burns: journal of the International Society for Burn Injuries 42(8) (2016) 1704-1711.

[11] S. A. Voils, S. J. Lemon, J. Jordan, P. Riley, R. Frye, Early thrombin formation capacity in trauma patients and association with venous thromboembolism, Thrombosis research 147 (2016) 13-15.

[12] W. H. Geerts, D. Bergqvist, G. F. Pineo, J. A. Heit, C. M. Samama, M. R. Lassen, C. W. Colwell, Prevention of venous thromboembolism: American College of Chest Physicians Evidence-Based Clinical Practice Guidelines (8th Edition), Chest 133(6 Suppl) (2008) 381S-453S.

[13] C. A. Karcutskie, A. Dharmaraja, J. Patel, S. A. Eidelson, A. G. Martin, E.B. Lineen, N. Namias, C. I. Schulman, K. G. Proctor, Relation of antifactor-Xa peak levels and venous thromboembolism after trauma, The journal of trauma and acute care surgery 83(6) (2017) 1102-1107.

[14] T. W. Costantini, E. Min, K. Box, V. Tran, R. D. Winfield, D. Fortlage, J. Doucet, V. Bansal, R. Coimbra, Dose adjusting enoxaparin is necessary to achieve adequate venous thromboembolism prophylaxis in trauma patients, The journal of trauma and acute care surgery 74(1) (2013) 128-33; discussion 134-5.

[15] D. Malinoski, F. Jafari, T. Ewing, C. Ardary, H. Conniff, M. Baje, A. Kong, M. E. Lekawa, M. O. Dolich, M. E. Cinat, C. Barrios, D. B. Hoyt, Standard prophylactic enoxaparin dosing leads to inadequate anti-Xa levels and increased deep venous thrombosis rates in critically ill trauma and surgical patients, The Journal of trauma 68(4) (2010) 874-80.

[16] C. A. Karcutskie, A. Dharmaraja, J. Patel, S. A. Eidelson, A. B. Padiadpu, A. G. Martin, G. Lama, E. B. Lineen, N. Namias, C. I. Schulman, K. G. Proctor, Association of Anti-Factor Xa-Guided Dosing of Enoxaparin With Venous Thromboembolism After Trauma, JAMA surgery 153(2) (2018) 144-149.

[17] A. H. Sabbagh, G. K. Chung, P. Shuttleworth, B. J. Applegate, W. Gabrhel, Fresh frozen plasma: a solution to heparin resistance during cardiopulmonary bypass, The Annals of thoracic surgery 37(6) (1984) 466-8.

[18] M. Kanbak, B. Oc, M. A. Salman, T. Ocal, M. Oc, Peroperative effects of fresh frozen plasma and antithrombin III on heparin sensitivity and coagulation during nitroglycerine infusion in coronary artery bypass surgery, Blood coagulation & fibrinolysis: an international journal in haemostasis and thrombosis 22(7) (2011) 593-9.

[19] D. du Cheyron, B. Bouchet, C. Bruel, C. Daubin, M. Ramakers, P. Charbonneau, Antithrombin supplementation for anticoagulation during continuous hemofiltration in critically ill patients with septic shock: a case-control study, Critical care 10(2) (2006) R45.

[20] J. T. Owings, M. Bagley, R. Gosselin, D. Romac, E. Disbrow, Effect of critical injury on plasma antithrombin activity: low antithrombin levels are associated with thromboembolic complications, The Journal of trauma 41(3) (1996) 396-405; discussion 405-6.

[21] J. T. Owings, R. Gosselin, Acquired antithrombin deficiency following severe traumatic injury: rationale for study of antithrombin supplementation, Seminars in thrombosis and hemostasis 23 Suppl 1 (1997) 17-24.

[22] G. T. Gerotziafas, A. D. Petropoulou, E. Verdy, M. M. Samama, I. Elalamy, Effect of the anti-factor Xa and anti-factor IIa activities of low-molecular-weight heparins upon the phases of thrombin generation, J Thromb Haemost 5(5) (2007) 955-62.

[23] S. Ben-Hadj-Khalifa, N. Hezard, W. Y. Almawi, M. G. Remy, B. Florent, T. Mahjoub, P. Nguyen, Differential coagulation inhibitory effect of fondaparinux, enoxaparin and unfractionated heparin in cell models of thrombin generation, Blood coagulation & fibrinolysis: an international journal in haemostasis and thrombosis 22(5) (2011) 369-73.

[24] M. Cini, C. Legnani, S. Testa, A. Tripodi, B. Cosmi, G. Palareti, An in vitro study to investigate the interference of enoxaparin on plasma levels of direct oral factor Xa inhibitors measured by chromogenic assays, Int J Lab Hematol 41(3) (2019) 309-315.

[25] J. C. Cardenas, A. P. Cap, M. D. Swartz, P. Huby Mdel, L. A. Baer, N. Matijevic, B. A. Cotton, J. B. Holcomb, C. E. Wade, Plasma Resuscitation Promotes Coagulation Homeostasis Following Shock-Induced Hypercoagulability, Shock 45(2) (2016) 166-73.

[26] T. Iba, A. Kidokoro, High-dose antithrombin therapy for sepsis: mechanisms of action, Shock 18(5) (2002) 389-94.

[27] B. H. McCully, C. R. Connelly, K. A. Fair, J. B. Holcomb, E. E. Fox, C. E. Wade, E. M. Bulger, M. A. Schreiber, P. S. Group, Onset of Coagulation Function Recovery Is Delayed in Severely Injured Trauma Patients with Venous Thromboembolism, Journal of the American College of Surgeons 225(1) (2017) 42-51.

[28] B. D. Spiess, Treating heparin resistance with antithrombin or fresh frozen plasma, The Annals of thoracic surgery 85(6) (2008) 2153-60.

1. A method for reducing the occurrence of a thrombosis or a thromboembolism in a patient identified as being at risk thereof, the method comprising administering to the patient a therapeutically effective amount of:
an anticoagulant selected from the group consisting of unfractionated heparin, a low molecular weight heparin, a heparinoid, fondaparinux, idraparinux, and combinations thereof, and
antithrombin III (ATIII).

2. The method according to claim 1, wherein said thrombosis is venous thrombosis.

3. The method according to claim 1, wherein said thromboembolism is a venous thromboembolism (VTE).

4. The method according to claim 1, wherein the patient identified as being at risk is selected from the group consisting of a physical trauma patient, a perioperative patient, a peripartum patient, patients with restricted mobility, cancer patients, sepsis patients, systemic inflammatory response syndrome patients, and combinations thereof.

5. The method according to claim 1, wherein the patient is a physical trauma patient, and the patient has been subjected to blunt force trauma, penetrating trauma, or a combination thereof.

6. The method according to claim 1, wherein the ATIII is administered at a concentration that increases the patient's ATIII levels to greater than about 1.0 IU/mL.

7. The method according to claim 1, wherein the ATIII is administered at a concentration that increases the patient's ATIII levels to greater than about 1.2 IU/mL.

8. The method according to claim 1, wherein the ATIII is administered at a concentration that increases the patient's ATIII levels to greater than about 1.3 IU/mL.

9. The method according to claim 1, wherein the ATIII is administered at a concentration that increases the patient's ATIII levels to greater than about 1.4 IU/mL.

10. The method according to claim 1, wherein the ATIII is administered at a concentration that increases the patient's ATIII levels to greater than about 1.5 IU/mL.

11. The method according to claim 1, wherein the ATIII is administered at a concentration that increases the patient's ATIII levels to a range of about 1.2 IU/mL to about 2.5 IU/mL.

12. The method according to claim 1, wherein the ATIII is administered at a concentration that increases the patient's ATIII levels to a range of about 1.5 IU/mL to about 2.0 IU/mL.

13. The method according to claim 1, wherein the ATIII is administered at a concentration that increases the patient's ATIII levels to a range of about 1.0 IU/mL to about 1.5 IU/mL.

14. (canceled)

15. The method according to claim 1, wherein the low molecular weight heparin is selected from the group consisting of Bemiparin, Certoparin, Dalteparin, Enoxaparin, Nadroparin, Parnaparin, Reviparin, Tinzaparin, combinations thereof, and pharmaceutically acceptable salts thereof.

16. The method according to claim 1, wherein the heparinoid is selected from the group consisting of Danaparoid, Dermatan sulfate, Sulodexide, combinations thereof, and pharmaceutically acceptable salts thereof.

17. (canceled)

18. The method according to claim 1, wherein the low molecular weight heparin is Enoxaparin or a pharmaceutically acceptable salt thereof.

19. The method according to claim 1, wherein the therapeutically effective amount of the low molecular weight heparin is a daily dose of about 20 mg to about 180 mg.

20. The method according to claim 1, wherein said therapeutically effective amount of the low molecular weight heparin is a daily dose of about 20 mg to about 40 mg.

21. The method according to claim 1, wherein said therapeutically effective amount of the low molecular weight heparin is from about 0.1 to about 2.5 mg/kg.

22. The method according to claim 1, wherein said therapeutically effective amount of the low molecular weight heparin is from about 0.5 to about 1.5 mg/kg.

23-44. (canceled)
